(19) United States
(12) Patent Application Publication
(10) Pub. No.: US 2024/0299681 A1
(43) Pub. Date: Sep. 12, 2024

(54) PROCESS FOR FLUSHING A GAS MEASUREMENT LINE OF A MEDICAL DEVICE, DEVICE, MEDICAL DEVICE, COMPUTER PROGRAM PRODUCT AND COMPUTER-READABLE MEDIUM

(71) Applicant: Drägerwerk AG & Co. KGaA, Lübeck (DE)

(72) Inventors: Thomas ROSSEN, Lübeck (DE); Bastian SCHULDT, Lübeck (DE)

(21) Appl. No.: 18/595,565

(22) Filed: Mar. 5, 2024

(30) Foreign Application Priority Data
Mar. 7, 2023 (DE) 10 2023 105 526.7

(51) Int. Cl. A61M 16/00

(52) U.S. Cl. CPC A61M 16/0003; A61M 16/0057; A61M 16/024

(57) ABSTRACT

A process for flushing a gas measurement line of a medical device includes the steps of: providing a first flushing volume flow to purge the gas sensing line toward a patient during a first time interval, and interrupting the first flushing volume flow during a second time interval. Further disclosed are a corresponding apparatus, a medical device, a computer program product with a computer readable medium.

### CROSS REFERENCE TO RELATED APPLICATIONS

This application claims the benefit of priority under 35 U.S.C. § 119 of German Application 10 2023 105 526.7, filed Mar. 7, 2023, the entire contents of which are incorporated herein by reference.

### TECHNICAL FIELD

The present invention relates to a process for flushing (purging) a gas measurement line of a medical device, a device, a medical device, a computer program product and a computer-readable medium.

### BACKGROUND

In some medical devices, in particular in ventilators and anesthesia machines, gas measurement lines can be provided to monitor a gas parameter, such as a ventilation parameter, of a therapy process such as a ventilation process. An example of such a ventilation parameter is airway pressure.

For example, the therapy form of invasive high-frequency ventilation is known to maintain an adequate supply of oxygen to the lungs in critical situations. During high-frequency ventilation, there are rapid changes in the volume flow and pressure of the applied ventilation volume flow, which makes it difficult to calculate an airway pressure based on the device's internal sensors. It is therefore known to provide a pressure measurement line arranged close to the patient in order to measure the airway pressure close to the patient.

Non-invasive ventilation is another example of a form of therapy in which pressure measurement lines are usually provided close to the patient. By providing the pressure measurement line close to the patient, synchronized ventilation can be achieved in an advantageous way.

There is a general risk that potentially contaminated respiratory gas from the patient and/or condensates can reach the medical device through a gas measurement line, especially through a pressure measurement line as described above. In particular, forms of therapy with pressure changes are a favorable factor for this type of respiratory gas reaching the medical device. However, even with forms of therapy without pressure changes, germs and/or condensates can migrate undesirably along the gas measurement line due to diffusion.

In order to prevent both the formation of condensate and the migration of germs through the gas measurement line in the direction of the medical device, it is known to dose a constant volume flow from the direction of the medical device in the direction of the patient, which acts as a flushing volume flow (purge volume flow/rinsing volume flow). For this measure to be effective, a sufficiently high flow rate of the flushing volume flow is required.

However, dosing a flushing volume flow has an undesirable effect on the breathing gas mixture on the patient.

### SUMMARY

It is an object of the invention to provide an improved process for flushing a gas measurement line of a medical device, a device, a medical device, a computer program product and a computer-readable medium, with which an undesirable influence on a therapy by flushing the gas measurement line can be reduced.

These and other objects are attained by the process for flushing a gas measurement line of a medical device, as well as a device practicing the process, a medical device, a computer program product and a computer-readable medium with features according to the invention.

This disclosure presents advantageous embodiments of the invention.

According to the invention, a process for flushing a gas measurement line (in particular a pressure measurement line) of a medical device (in particular a ventilator and/or an anesthesia device) is provided in this respect. The process comprises the steps of: providing a first flushing volume flow for flushing (purging) the gas measurement line in the direction of a patient during a first time interval and interrupting the first flushing volume flow during a second time interval.

According to the invention, a first flushing volume flow is thus provided, which is not applied over the entire duration of the process, but only during a first time interval. In other words, a constant flushing volume flow is not provided over the duration of the process, but rather a flushing volume flow that is only provided in phases. During the first time interval, the first flushing volume flow therefore leads to flushing of the gas measurement line. However, by interrupting during the second time interval, a mean flow rate averaged over the duration of the process can advantageously be reduced compared to constantly provided flushing volume flows. This results in a reduced influence on the therapy, in particular a reduced influence on the inspiratory oxygen concentration.

In this respect, a gas measurement line is understood to be a line that directly or indirectly connects a medical device or a device and a patient or a patient interface in terms of flow and is thus set up to conduct gas. A number of sensors can be provided in or on the gas measurement line or in or on the medical device in order to determine one or more parameters of the conducted gas, such as the airway pressure of a ventilated patient.

A medical device is understood to be a device for use in or for a medical procedure, in particular for a therapeutic and/or a surgical procedure. Preferably, a medical device is understood to be a ventilator and/or an anesthesia device.

A flushing volume flow is understood to be a volume flow for at least temporarily flushing a number (i.e. singular or plural) of previously described gas measurement lines in the direction of a patient.

In the context of the invention, a flushing volume flow is characterized by a volume flow, which is also referred to below as a flow rate. In the context of the invention, each volume flow or flow rate, unless otherwise specified, can be essentially constant during its provision or can be variable over time.

In the context of the invention, a time interval is understood to be a time window with a specific duration and a specific position on a time axis. The time interval is greater than zero. The position can be relative to an absolute zero point of the time axis. In a preferred embodiment of the invention, the process can be performed repetitively during a predetermined number of periods or cycles (i.e. periodic or cycled), each cycle being characterized by a cycle duration. In this case, the position of the time interval may be determined relative to the beginning of a cycle. In the context of the invention, the predetermined number of cycles may be a singular or a plurality.

In the event that the process is carried out periodically cyclically, it is preferred that the first flushing volume flow (and each further flushing volume flow or basic volume flow to be described) has a flow rate that is the same for each cycle.

Preferably, the process further comprises the steps of: receiving a first piece of information (first information) corresponding to a flow rate of the first flushing volume flow, receiving a second piece of information (second information) corresponding to a volume of the gas measurement line, and determining a first duration of the first time interval depending on the first information and on the second information.

In this way, the first duration of the first time interval can be determined particularly easily, since the first information and the second information can be determined in a simple manner.

A lower threshold of the first time duration is particularly preferred in this configuration. This is a particularly effective way of ensuring that the volume of the gas measurement line has been flushed (purged) at least once.

In the context of the invention, the information (i.e. the first information and/or the second information and/or further third, fourth and fifth information to be described) may be stored or may be storable in a memory unit, for example of a medical device or a device, and thus may be received during the process, for example by a control unit.

The first piece of information can, for example, be provided as an actual flow rate by measuring the flow rate of the flushing volume flow in the gas measurement line. In addition or alternatively, the first piece of information can, for example, be available as predetermined first information and correspond, for example, to a target flow rate or an expected flow rate.

The second piece of information can be obtained, for example, by calibration, from a data sheet for the gas sampling line or by calculation using geometric parameters of the gas sampling line. A set of information can also be received as second information, such as the length and diameter of the gas measurement line, from which the volume of the gas measurement line can be determined.

In the context of the invention, the first time duration (and each further time duration still to be described) can be determined—for example by a control unit—by processing a calculation rule stored in a memory unit. The first time duration (and each further time duration still to be described) can be determined—for example by a control unit—additionally or alternatively by reading table values from a table stored in a memory unit. Such a table can, for example, be available as a lookup table.

Preferably, the process further comprises the steps of: determining a second duration of the second time interval as a function of the first duration, such that the second duration is at least as large as the first duration.

In this way, an undesirable influence on a therapy can be advantageously further reduced by ensuring that the first flushing volume flow is no longer provided by being interrupted. In this way, it can be reliably achieved that a respiratory gas composition on the patient returns to a desired target state before a new cycle (period) of the procedure.

Preferably, the process further comprises the steps of: providing a second flushing volume flow in the gas measurement line in the direction of the patient, wherein preferably an oxygen concentration of the first flushing volume flow and an oxygen concentration of the second flushing volume flow are different from each other.

An undesirable influence on the therapy, in particular the inspiratory oxygen concentration, can be advantageously reduced in this preferred embodiment. In this embodiment, the composition of the respiratory gas is additionally influenced by the second flushing volume flow, so that there is a greater degree of freedom in influencing the respiratory gas composition by suitable selection of the supplied quantities of first flushing volume flow and second flushing volume flow.

For example, the first flushing volume flow or the second flushing volume flow may comprise air or consist of air and the second flushing volume flow or the first flushing volume flow may comprise an air/oxygen mixture or pure oxygen or consist of an air/oxygen mixture or pure oxygen.

Preferably, the process further comprises the steps of: receiving a third piece of information (third information) corresponding to an oxygen concentration of a ventilation volume flow, receiving a fourth piece of information (fourth information) corresponding to an oxygen concentration of the first flushing volume flow, optionally receiving a fifth piece of information (fifth information) corresponding to an oxygen concentration of the second flushing volume flow, and determining the first time duration depending on the third information, from the fourth information and optionally from the fifth information, so that the inspiratory oxygen concentration does not exceed or fall below a threshold value by mixing the ventilation volume flow, the first flushing volume flow and the optional second flushing volume flow.

In this way, the first time duration can be provided in a particularly advantageous way, as the third information and the fourth information can be determined easily. Furthermore, this ensures that the success of the therapy is not impaired.

In this embodiment, it is particularly preferable to determine an upper threshold for the first duration. In this way, it can be ensured particularly effectively that an influence on the therapy by the flushing does not exceed or fall below a certain threshold value, in particular that an inspiratory oxygen concentration does not exceed or fall below a certain amount.

A ventilation volume flow is a volume flow of a gas that the medical device provides to ventilate the patient.

An oxygen concentration of the ventilation volume flow is understood to mean a setpoint value of an oxygen concentration of the ventilation volume flow and/or an actual value of an oxygen concentration of the ventilation volume flow. A setpoint value of the oxygen concentration of the ventilation volume flow can, for example, be predetermined by a gas supply of the medical device and be essentially unchangeable. An actual value of the oxygen concentration of the ventilation volume flow can, for example, be determined by suitable sensors.

The ventilation volume flow and the flushing volume flow mix at the patient (at or near the patient interface) to form a breathing gas with which the patient is ventilated.

For example, a maximum or minimum permissible inspiratory oxygen concentration can be specified as a threshold value, for example taking into account a permissible deviation of 5% by volume from a target value.

Preferably, the process further comprises the steps of: providing the second flushing volume flow during a third time interval, wherein the third time interval corresponds at least to the second time interval, and interrupting the second flushing volume flow during a fourth time interval, wherein the fourth time interval corresponds at most to the first time interval.

A time interval corresponding to another time interval denotes a time interval which is identical to the other time interval in terms of position and duration. A time interval corresponding to at least one other time interval thus denotes a time interval which is identical to the other time interval in terms of position and duration and may additionally have a further extension beyond the other time interval. A time interval corresponding to at most one other time interval thus denotes a time interval which is limited by the other time interval in terms of position and duration and can have a shorter duration than the other time interval within these limits.

In this way, the first flushing volume flow and the second flushing volume flow can be provided immediately adjacent to each other or overlapping in terms of time, so that at least one of the two flushing volume flows is provided at any time during the process duration or cycle. The risk of germs or condensate migrating through the gas measurement line can thus be effectively reduced.

In the preferred case that the first flushing volume flow and the second flushing volume flow are provided overlapping in terms of time, a combined flushing volume flow with a correspondingly increased flow rate is achieved for the period of overlap. By specifically selecting the overlap duration and respective flow rate of the first flushing volume flow and the second flushing volume flow, providing an intermittent flushing volume flow can be achieved, which ensures particularly effective flushing due to the temporarily increased flow rate.

Preferably, the process further comprises the steps of: providing the second flushing volume flow during the second time interval, and interruption of the second flushing volume flow during the first time interval.

In this way, the first flushing volume flow and the second flushing volume flow can be provided immediately adjacent to each other in terms of time, so that at least one of the two flushing volume flows is provided at any time during the process duration or cycle time. The risk of germs or condensate migrating through the gas measurement line can thus be effectively reduced.

Preferably, the process further comprises the step of: providing a continuous basic volume flow in a breathing gas line of the medical device during the execution (in particular during the cycle time or period) of the process.

Continuous provision means that the basic volume flow is provided continuously during the process.

In this way, at least the basic volume flow is provided at all times during the duration of the procedure or cycle time. The mixture of basic volume flow with the first flushing volume flow and optionally the second flushing volume flow achieved in this way on the patient can reduce falsification of the ventilation parameters, in particular the inspiratory oxygen concentration.

The basic volume flow can have a constant flow rate or a variable flow rate during its provision.

In general, further flushing volume flows can be provided within the scope of the invention in addition to the first flushing volume flow and the optional second flushing volume flow.

According to the invention, a device is also provided which is configured to carry out at least one of the processes described above. The device has a first flushing gas source (purge gas source) for providing the first flushing volume flow and optionally a second flushing gas source for providing the second flushing volume flow. In addition or alternatively, the device can be connected to (or has a connection configured to connect to) the first flushing gas source and optionally connected to (or has a connection configured to connect to) the second flushing gas source.

The device provides advantages and effects comparable to the process according to the invention.

In this respect, a flushing gas source is understood to be a unit that is set up to emit flushing gas. Flushing gas is understood to be a gas or a gas mixture that is suitable for flowing through the gas measurement line and reaching the patient. For example, the flushing gas may comprise air and/or oxygen or consist of air and/or oxygen.

One or both flushing gas sources can be configured as stationary units, for example a central gas supply of a hospital. One or both flushing gas sources can, additionally or alternatively, be configured as transient units, such as pressurized gas containers, blowers, fans and/or centrifugal compressors.

The device can, for example, be configured as a module separate from a medical device, which can be connected to the medical device (mechanically, by data, electrically and/or fluidically). In this case, the device can be suitable for retrofitting or for extending the functionality of a medical device.

The device can also be configured, for example, as a unit that can be integrated into a medical device.

The device may have one or more mechanical, data, electrical and/or fluidic interfaces to connect the device to one or both flushing gas sources, the gas measurement line and/or optionally the medical device and optionally a control unit and storage unit.

According to the invention, a medical device is also provided. The medical device has the device described above and a gas measurement line for carrying out at least one process described above.

The device and/or the medical device may comprise, as an integral part or connected via an interface, a control unit for carrying out at least one process according to the invention. The device and/or the medical device may further comprise a memory unit for providing the above-described information.

Preferably, the gas measurement line is configured as a pressure measurement line, in particular as a pressure measurement line close to the patient. Additionally or alternatively, the medical device is preferably configured as a ventilator or as an anesthesia device.

Preferably, the medical device can have a pressure sensor and a pressure relief valve that can be opened to the environment of the medical device to record a pressure course (pressure curve) on the patient. The pressure relief valve can be set up to open to the environment from a certain pressure above a measuring range of the pressure sensor but below a destruction threshold of the pressure sensor. In this state, the pressure relief valve is set up to direct the first flushing volume flow and optionally the second flushing volume flow into the environment in order to prevent destruction of the overpressure sensor, in particular in the event of a stenosis in the gas measurement line.

Preferably, the process also has the step of: performing an offset calibration, whereby the offset calibration makes it possible to determine a pressure drop caused by applying the first flushing volume flow and optionally the second flushing volume flow to the gas measurement line as a (constant or variable, for example time-dependent) predetermined offset value. The offset value determined in this way can be corrected in an evaluation of the ventilation parameters monitored by the gas measurement line.

According to the invention, a computer program product is further provided. The computer program product comprises instructions that cause the device and/or the medical device to perform at least one of the above-described processes according to the invention.

According to the invention, a computer-readable medium is further provided on which the computer program product is stored. The computer readable storage medium can be a tangible device that is non-transitory and that can retain and store instructions for use by an instruction execution device such as the control unit. The computer readable storage medium may be, for example, but is not limited to, an electronic storage device, a magnetic storage device, an optical storage device, an electromagnetic storage device, a semiconductor storage device, or any suitable combination of the foregoing.

These and other features and effects of the invention are also apparent from the following description of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic flow rate versus time diagram of an embodiment of a process according to the invention;

FIG. 2 is a flow chart of an embodiment of a process according to the invention;

FIG. 3 is a schematic representation of an embodiment of a device according to the invention and a medical device according to the invention;

FIG. 4 is a flow chart of an embodiment of a process according to the invention;

FIG. 5 is a flow chart of an embodiment of a process according to the invention;

FIG. 6 is a flow chart of an embodiment of a process according to the invention;

FIG. 7 is a flow chart of an embodiment of a process according to the invention;

FIG. 8 is a flow chart of an embodiment of a process according to the invention;

FIG. 9 is a flow chart of an embodiment of a process according to the invention;

FIG. 10 is a flow chart of an embodiment of a process according to the invention;

FIG. 11 is a schematic flow rate versus time diagram of an embodiment of a process according to the invention;

FIG. 12 is a schematic volume flow-time diagram of an embodiment of a process according to the invention;

FIG. 13 is a schematic flow rate versus time diagram of an embodiment of a process according to the invention; and

FIG. 14 is a schematic volume flow-time diagram of an embodiment of a process according to the invention.

### DESCRIPTION OF PREFERRED EMBODIMENTS

According to the invention, processes for flushing a gas measurement line 10 of a medical device 40, a device 30, a medical device 40, a computer program product and a computer-readable medium are provided.

An embodiment example of a medical device 40 according to the invention is shown in FIG. 3. A medical device 40, configured as a ventilator or as an anesthesia device in the illustrated embodiment example, can have a breathing gas line 20 such as a breathing tube (hose) for ventilating a patient P via a patient interface not shown in detail. The breathing tube 20 can have an inspiratory branch and an expiratory branch. A patient interface can be, for example, a nasal cannula (prongs), a breathing mask or a tube.

The breathing tube 20 or the patient interface can be connected or connectable to a gas measurement line 10. The gas measurement line 10 can, for example, be configured as a fluid guide tube. The gas measurement line 10 can be fluidically connected to an area of the breathing tube 20 or the patient interface that is close to the patient. In this respect, an area close to the patient is an area that is located closer to the patient P than to the medical device 40.

According to the invention, a device 30 is provided, which in FIG. 3 is formed as part of the medical device 40, for example as an assembly of the medical device 40 or as a module of the medical device 40. However, in an example not shown, the device 30 may be provided separately from the medical device 40.

The gas measurement line 10 can be in fluidic connection with a sensor unit 70 to determine parameters to be monitored, such as ventilation parameters. The sensor unit 70 can, for example, as shown, be a structural part of the medical device 40 or, in an embodiment example not shown, a structural part of the device 30.

For example, the gas measurement line 10 can be configured as a pressure measurement line in the embodiment example shown and the sensor unit 70 can be configured as a pressure sensor.

The gas measurement line 10 can be connected to the medical device 40 via a third interface 41. The gas measurement line 10 can be connected to the device 30 via a first interface 36. In the illustrated embodiment example, the gas measurement line 10 can be connectable first to the medical device 40 via the third interface 41 and then to the device 30 via the first interface 36.

The sensor unit 70 can be connected to the device 30 and the gas measurement line 10 via a second interface 37 and a line 42.

The gas measurement line 10 can be an integral part of the medical device 40 or can be configured as an exchangeable component of the medical device 40, for example for single use.

The medical device 40 and/or the device 30—in the illustrated embodiment example the medical device 40—can have a control unit 50 and a memory unit 60. The control unit 50 can be configured, by means of suitable hardware and/or software, to control the device 30 and thus to carry out the process according to the invention. The control unit 50 can also be set up to receive and evaluate measured values from the sensor unit 70.

The device 30 is configured to carry out the process according to the invention. In the embodiment example shown, the device 30 has a first flushing gas source (purge gas source) 31 for providing a first flushing volume flow v1 and optionally a second flushing gas source 32 for providing the second flushing volume flow v2. In an embodiment example not shown, the device can instead have a connection to a first flushing gas source 31 for providing a first flushing volume flow v1 and optionally have a connection to a second flushing gas source 32 for providing the second flushing volume flow v2.

The first flushing gas source 31 is connectable to the first interface 36 via a line 34. The line 34 may have valves or other actuators, not shown, for selectively closing the line 34. The valves or other actuators may be controllable by the control unit 50.

The second flushing gas source 32 is connectable to the first interface 36 via a conduit 33. The line 33 may include valves or other actuators, not shown, for selectively closing the line 33. The valves or other actuators may be controllable by the control unit 50.

Line 33 and line 34 can be connected by a mixer (e.g. configured as a T-piece) and/or by a valve.

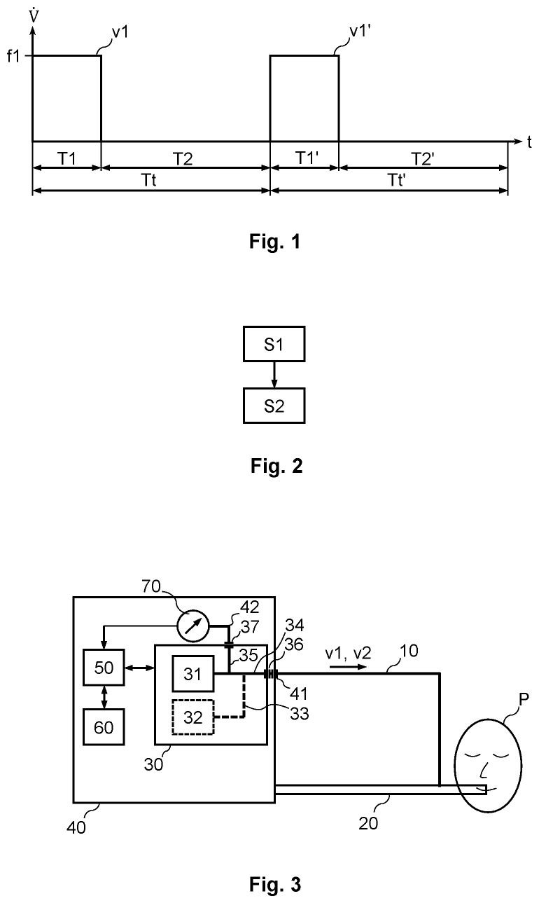

FIG. 1 shows a schematic volume flow rate V-time t diagram of an embodiment example of a process according to the invention for flushing the gas measurement line 10 of the medical device 40 is shown. FIG. 2 shows a corresponding flow diagram of the embodiment example according to FIG. 1.

The process according to FIGS. 1 and 2 comprises the steps S1: providing a first flushing volume flow v1 for flushing the gas measurement line 10 in the direction of the patient P during a first time interval T1 and S2: interrupting the first flushing volume flow v1 during a second time interval T2.

The first flushing volume flow v1 is characterized by a first flow rate f1. The flow rate f1 can be constant or variable over time during the provision of the first flushing volume flow v1. In the illustrated embodiment example according to FIG. 1, the first flow rate f1 is constant during its provision.

In the illustrated embodiment example, it is provided that the process steps S1 and S2 are carried out repeatedly for a predetermined number of periods or cycles (i.e. periodically, cyclically). Each cycle of a repetition is characterized by a cycle duration Tt or Tt′. The predetermined number of cycles can be a single number or a plurality. In the example shown in FIG. 1, two cycles are shown.

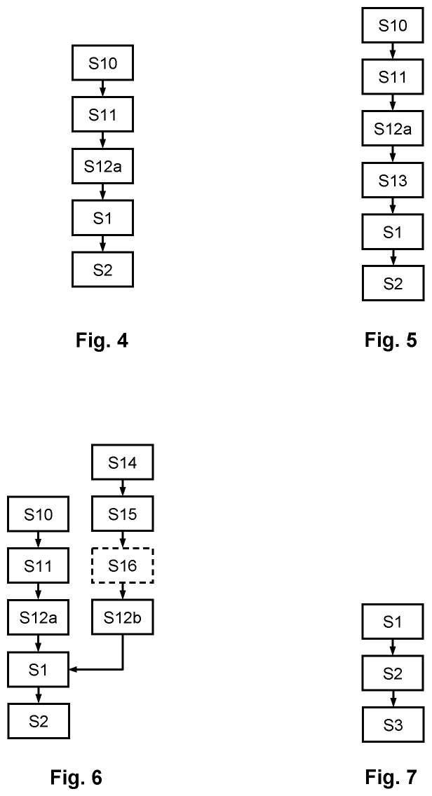

FIG. 4 shows a flow chart of a modification of the process shown in FIGS. 1 and 2.

The process according to FIG. 4 further comprises the steps: S10 receiving first information I1 corresponding to a flow rate f1 of the first flushing volume flow v1, S11 receiving second information I2 corresponding to a volume VL of the gas measurement line 10, and S12a determining a first time duration t1 of the first time interval T1 as a function of the first information I1 and of the second information I2.

A lower threshold t1min of the first time duration t1 is particularly preferably determined in this way.

The first information I1 may be stored or storable in the memory unit 60 and thus received by the control unit 50 when the process is performed.

For example, the first time duration t1, preferably the lower threshold t1min of the first time duration t1, can be determined using the following formula:

$$t 1 = VL / f 1.$$

FIG. 5 shows a flow chart of a modification of the process shown in FIG. 4.

The process according to FIG. 5 corresponds to the process according to FIG. 4, but additionally has the step: S13 determining a second time duration t2 of the second time interval T2 as a function of the first time duration t1, so that the second time duration t2 is at least as long as the first time duration t1.

For example, the second time duration t2 can be determined using the following formula:

t2≥t1.

FIG. 7 shows a flow chart of a modification of the process shown in FIG. 1.

The process according to FIG. 7 corresponds to the process according to FIG. 1, but additionally has the step: S3 providing a second flushing volume flow v2 in the gas measurement line 10 in the direction of the patient P, wherein preferably an oxygen concentration c1 of the first flushing volume flow v1 and an oxygen concentration c2 of the second flushing volume flow v2 are different from each other.

The processes according to FIGS. 4, 5 and 6 can also have the step S3 described in connection with FIG. 7.

A schematic volume flow V-time t diagram of the process according to FIG. 7 is shown in FIG. 11.

The diagram shown in FIG. 11 is a modification of the diagram in FIG. 1. The diagram also shows the second flushing volume flow v2. The first flushing volume flow v1 and the second flushing volume flow v2 can overlap in time or—as shown in FIG. 11—not overlap in time. For example, the second flushing volume flow v2 can be provided during a third time interval T3 and interrupted during a fourth time interval T4, whereby the third time interval T3 can correspond at most to the second time interval T2.

The first flushing volume flow v1 is characterized by a first flow rate fL. The first flushing volume flow v2 is characterized by a second flow rate f2. The first flow rate f1 and/or the second flow rate f2 can be constant or variable over time during the provision of the first flushing volume flow v1 or the second flushing volume flow f2. In the illustrated embodiment example, the provided first flow rate f1 and second flow rate f2 are constant.

In the embodiment example shown, it is envisaged that the process steps described are carried out repeatedly for a predetermined number of periods or cycles (i.e. periodically, cyclically). Each cycle of a repetition is characterized by a cycle duration Tt or Tt′. The predetermined number of cycles can be a single number or a plurality. In the embodiment example, two cycles are shown.

FIG. 6 shows a flow chart of a modification of the process shown in FIG. 4.

The process according to FIG. 6 corresponds to the process according to FIG. 4, but additionally comprises the steps: S14 receiving a third piece of information I3, which corresponds to an oxygen concentration cB of a ventilation volume flow vB, S15 receiving a fourth piece of information I4, which corresponds to an oxygen concentration c1 of the first flushing volume flow v1, and S12b determining the first time duration t1 as a function of the third information I3 and of the fourth information I4, so that the inspiratory oxygen concentration cL does not exceed or fall below a threshold value C by mixing the ventilation volume flow vB and the first flushing volume flow v1.

In this embodiment, an upper threshold t1max of the first time duration t1 is particularly preferred

For example, the first time duration t1, preferably the upper threshold t1max of the first time duration t1, can be determined by iterative solution processes using the following system of equations:

$$cmix = ( fB \cdot cB + f 1 \cdot c 1 ) / ( fB + f 1 ) , \\ cL ( n ) = ( A \cdot cL ( n - 1 ) + B \cdot cmix ) / ( A + B ) , \\ cL ( n = n\_Threhold ) \leq C , \\ t 1 = ( n\_Threshold ) / F .$$

Here cmix denotes the oxygen concentration of the respiratory gas, n the nth breath within a period, A the functional residual capacity of the patient, B the tidal volume and C a threshold value of the inspiratory oxygen concentration. n_threshold denotes the breath within the period at which the threshold value C is not yet exceeded. F denotes the ventilation frequency with which the patient is ventilated.

The parameters A, B and F depend on the patient and ventilation mode and can be obtained by the control unit 50 as information from the medical device 40, by manual input by a user, by monitoring the ventilation process and/or from a database and stored in the memory unit 60 for further processing.

The threshold value C can be stored as predetermined information in the memory unit 60 and/or can be predetermined by manual input by a user.

The process according to FIG. 6 can also have the step S13 described in connection with FIG. 5. In this way, it can be ensured that the oxygen concentration cmix of the breathing gas is adjusted to the oxygen concentration of the ventilation gas volume flow at the end of the period.

The embodiment example shown in FIG. 6 may further comprise the optional step S16 of receiving a fifth piece of information (fifth information) I5 corresponding to an oxygen concentration c2 of the second flushing volume flow v2, wherein the step S12b in this case comprises: determining the first time duration t1 depending on the third information I3, on the fourth information I4 and on the fifth information I5, so that a threshold value C of the inspiratory oxygen concentration cL is not exceeded by mixing the ventilation volume flow vB, the first flushing volume flow v1 and the second flushing volume flow v2.

FIG. 8 shows a flow chart of a modification of the process in FIG. 7, which can be combined with any of the processes in FIGS. 2, 4, 5 and 6.

The above-described step S3 can thus comprise the steps: S31 providing the second flushing volume flow v2 during a third time interval T3, the third time interval T3 corresponding at least to the second time interval T2, and S41 interrupting the second flushing volume flow v2 during a fourth time interval T4, the fourth time interval T4 corresponding at most to the first time interval T1.

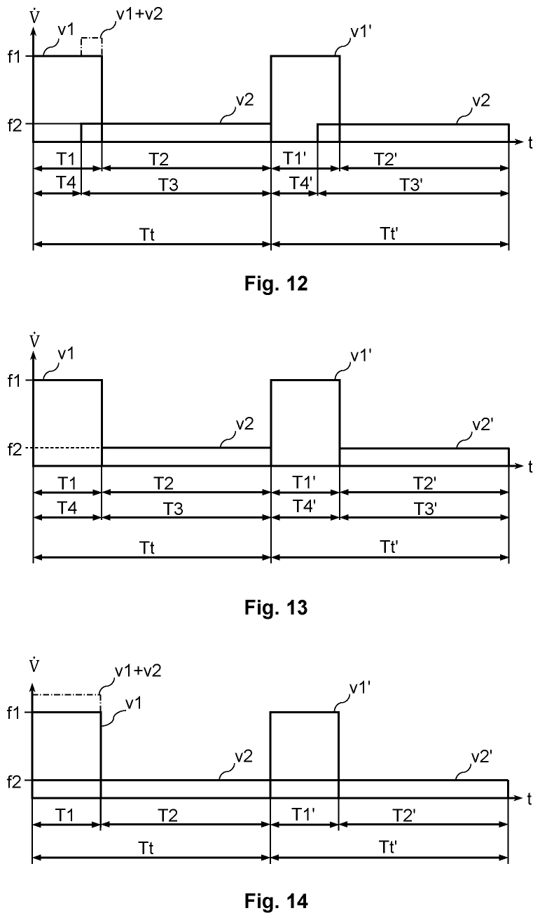

A schematic volume flow V-time t diagram of the process according to FIG. 8 is shown in FIG. 12. As can be seen in FIG. 12, the process is configured in such a way that the first volume flow v1 and the second volume flow v2 overlap in time and form a combined volume flow v1+v2 with an increased flow rate for the period of this overlap.

The first flushing volume flow v1 is characterized by a first flow rate f1. The first flushing volume flow v2 is characterized by a second flow rate f2. The first flow rate f1 and/or the second flow rate f2 can be constant or variable over time during the provision of the first flushing volume flow v1 or the second flushing volume flow f2. In the embodiment example shown, the provided first flow rate f1 and the provided second flow rate f2 are constant.

In the embodiment example shown, it is envisaged that the process steps described are carried out repeatedly for a predetermined number of periods cycles (i.e. periodically, cyclically). Each cycle of a repetition is characterized by a cycle duration Tt or Tt′. The predetermined number of cycles can be a single number or a plurality. In the embodiment example, two cycles are shown.

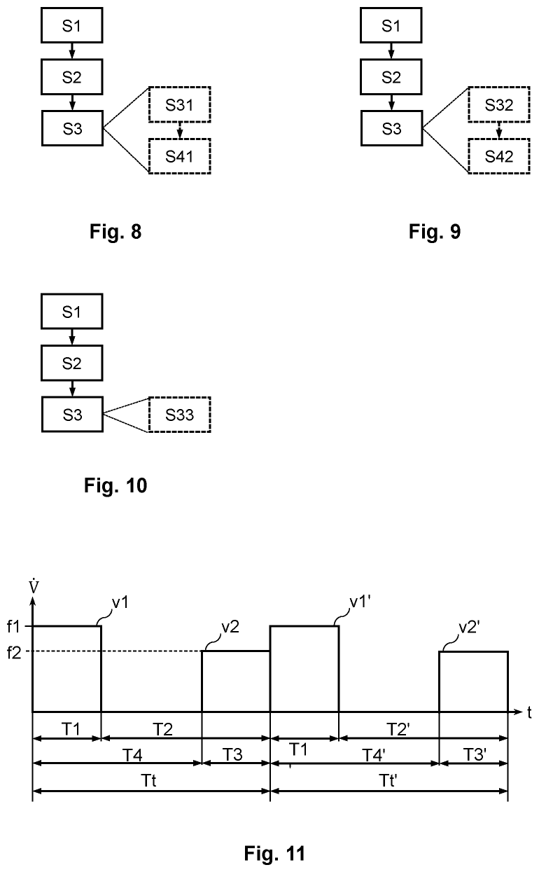

FIG. 9 shows a flow chart of a modification of the process in FIG. 7, which can be combined with any of the processes in FIGS. 2, 4, 5 and 6.

The above-described step S3 can thus comprise the steps: S32 providing the second flushing volume flow v2 during the second time interval T2, and S42 interrupting the second flushing volume flow v2 during the first time interval T1.

A schematic volume flow V-time t diagram of the process according to FIG. 9 is shown in FIG. 13. As can be seen in FIG. 13, the process is configured in such a way that the first volume flow v1 and the second volume flow v2 do not overlap in time.

The first flushing volume flow v1 is characterized by a first flow rate f1. The second flushing volume flow v2 is characterized by a second flow rate f2. The first flow rate f1 and/or the second flow rate f2 can be constant or variable over time during the provision of the first flushing volume flow v1 or the second flushing volume flow f2. In the embodiment example shown, the provided first flow rate f1 and the provided second flow rate f2 are constant.

In the embodiment example shown, it is envisaged that the process steps described are carried out repeatedly for a predetermined number of periods or cycles (i.e. periodically, cyclically). Each cycle of a repetition is characterized by a cycle duration Tt or Tt′. The predetermined number of cycles can be a single number or a plurality. In the embodiment example, two cycles are shown.

FIG. 10 shows a flow chart of a modification of the process according to FIG. 7, which can be combined with any of the processes according to FIGS. 2, 4, 5 and 6.

The above-described step S3 can thus comprise the step: S33 Providing a continuous basic volume flow in the breathing gas line 20 of the medical device 40 during the performance of the process.

A schematic volume flow \dot{V}-time t diagram of the process according to FIG. 10 is shown in FIG. 14. As can be seen in FIG. 14, the process is configured in such a way that the first volume flow v1 and the second volume flow v2 overlap in time and form a combined volume flow v1+v2 with an increased flow rate for the period of this overlap.

The first flushing volume flow v1 is characterized by a first flow rate f1. The second flushing volume flow v2 is characterized by a second flow rate f2. The first flow rate f1 and/or the second flow rate f2 can be constant or variable over time during the provision of the first flushing volume flow v1 or the second flushing volume flow f2. In the embodiment example shown, the provided first flow rate f1 and the provided second flow rate f2 are constant.

The second flow rate f2 of the second flushing volume flow v2 should be selected so that the oxygen concentration cmix resulting from mixing the ventilation gas volume flow vB, the first flushing volume flow v1 and the second flushing volume flow v2 does not exceed or fall below a predetermined threshold value C.

This can be achieved by determining the second flow rate f2, preferably an upper threshold of the second flow rate f2, according to the equation below:

$$f 2 = ( f 1 \cdot ( c 1 - C ) ) / ( C - cB ) .$$

Where cB is the oxygen concentration of the ventilation gas volume flow vB, f1 is the flow rate of the first flushing volume flow v1, c1 is the oxygen concentration of the first flushing volume flow v1, C is the threshold value and f2 is the second flow rate of the second flushing volume flow c2.

The threshold value C can be stored as predetermined information in the memory unit 60 and/or can be predetermined by manual input by a user.

In the embodiment example shown, it is envisaged that the process steps described are carried out repeatedly for a predetermined number of periods or cycles (i.e. periodically, cyclically). Each cycle of a repetition is characterized by a cycle duration Tt or Tt′. The predetermined number of cycles can be a single number or a plurality. In the embodiment example, two cycles are shown.

Not shown is that there is further provided a computer program comprising instructions that cause the pre-described device 30 and/or the pre-described medical device 40 to perform at least one or all of the illustrated and described processes.

It is not shown that a computer-readable medium is also provided on which the computer program product described above is stored. The computer readable storage medium can be a tangible device that is non-transitory and that can retain and store instructions for use by an instruction execution device such as the control unit 50.

While specific embodiments of the invention have been shown and described in detail to illustrate the application of the principles of the invention, it will be understood that the invention may be embodied otherwise without departing from such principles.

### LIST OF REFERENCE CHARACTERS

10 Gas measurement line
20 Breathing hose, breathing gas line
30 Device
31 First flushing gas source
32 Second flushing gas source
33 Second line
34 First line
35 Third line
36 First interface
37 Second interface
40 Medical device
41 Third interface
50 Control unit
60 Storage unit
70 Sensor unit
A Functional residual capacity
B Tidal volume
C Threshold value
c1 Oxygen concentration of the first flushing volume flow
c2 Oxygen concentration of the second flushing volume flow
cB Oxygen concentration of the ventilation volume flow
cL Target oxygen concentration of the ventilation volume flow
cmix Oxygen concentration of the breathing gas
F Ventilation frequency
f1 First flow rate
f2 Second flow rate
fb Flow rate of the ventilation volume flow
I First information
I2 Second information
I3 Third information
I4 Fourth information
I5 Fifth information
P Patient
S1, S2, . . . Process steps
T Time
t1 First duration (period)
t1min Upper threshold of the first duration
t1max Upper threshold of the first duration
t2 Second duration (period)
t3 Third duration (period)
t4 Fourth duration (period)
ts Rinsing time
T1 First time interval
T2 Second time interval
T3 Third time interval
T4 Fourth time interval
Tt Cycle duration
V Volume flow
v1 First flushing volume flow
v2 Second flushing volume flow
vB Ventilation volume flow
VL Volume of the gas measurement line

What is claimed is:

1. A process of flushing a gas measurement line of a medical device, the process comprising the steps of:
providing a first flushing volume flow for flushing the gas measurement line in a direction of a patient during a first time interval; and
interrupting the first flushing volume flow during a second time interval.

2. A process according to claim 1, further comprising the steps of:
receiving first information corresponding to a flow rate of the first flushing volume flow;
receiving second information corresponding to a volume of the gas measurement line; and
determining a first duration of the first time interval as a function of the first information and of the second information.

3. A process according to claim 1, further comprising the step of determining a second time duration of the second time interval as a function of the first duration, so that the second duration is at least as long as the first duration.

4. A process according to claim 1, further comprising the step of providing a second flushing volume flow in the gas measurement line in the direction of the patient.

5. A process according to claim 4, wherein an oxygen concentration of the first flushing volume flow and an oxygen concentration of the second flushing volume flow are different from each other.

6. A process according to claim 1, further comprising the steps of:
receiving third information that corresponds to an oxygen concentration of a ventilation volume flow;
receiving fourth information corresponding to an oxygen concentration of the first flushing volume flow; and
determining the first duration depending on the third information and on the fourth information, so that by mixing the ventilation volume flow, the first flushing volume flow does not exceed or fall below a threshold value of the inspiratory oxygen concentration.

7. A process according to claim 4, further comprising the steps of:
receiving third information that corresponds to an oxygen concentration of a ventilation volume flow;
receiving fourth information corresponding to an oxygen concentration of the first flushing volume flow;
receiving fifth information corresponding to an oxygen concentration of the second flushing volume flow; and
determining the first time duration depending on the third information, on the fourth information and on the fifth information, so that by mixing the ventilation volume flow, the first flushing volume flow and the second flushing volume flow do not exceed or fall below a threshold value of the inspiratory oxygen concentration.

8. A process according to claim 4, further comprising the steps of:
providing the second flushing volume flow during a third time interval, wherein the third time interval corresponds at least to the second time interval; and
interrupting the second flushing volume flow during a fourth time interval, wherein the fourth time interval corresponds at most to the first time interval.

9. A process according to claim 4, further comprising the steps of:
providing the second flushing volume flow during the second time interval; and
interrupting the second flushing volume flow during the first time interval.

10. A process according to claim 1, further comprising the step of providing a continuous basic volume flow in a breathing gas line of the medical device during the performance of the process.

11. A process according to claim 1, further comprising providing a computer program product with a computer readable storage medium comprising a tangible device that is non-transitory with instructions retained and stored on the computer readable storage medium for use by an instruction execution device which causes the device and/or the medical device to execute the process for flushing the gas measurement line of the medical device.

12. A device configured to flush a gas measurement line of a medical device, the device comprising:
means providing a first flushing volume flow for flushing the gas measurement line in a direction of a patient during a first time interval and interrupting the first flushing volume flow during a second time interval; and
a first flushing gas source connection or a first flushing gas source for the first flushing volume flow.

13. A device according to claim 12, in combination with a gas measurement line and a medical device.

14. A device according to claim 13, wherein the gas measurement line is configured as a pressure measurement line, and/or wherein the medical device is configured as a ventilator or as an anesthesia device.

15. A device according to claim 12, wherein a control unit configured to:
receive first information corresponding to a flow rate of the first flushing volume flow;
receive second information corresponding to a volume of the gas measurement line; and
determine a first duration of the first time interval as a function of the first information and of the second information.

16. A device according to claim 15, wherein the control unit is configured to determine a second time duration of the second time interval as a function of the first duration, so that the second duration is at least as long as the first duration.

17. A device according to claim 15, further comprising means providing a second flushing volume flow in the gas measurement line in the direction of the patient and interrupting the second flushing volume flow.

18. A device according to claim 15, wherein the control unit is configured to:
receive third information that corresponds to an oxygen concentration of a ventilation volume flow;
receive fourth information corresponding to an oxygen concentration of the first flushing volume flow; and
determine the first duration depending on the third information and on the fourth information, so that by mixing the ventilation volume flow, the first flushing volume flow does not exceed or fall below a threshold value of the inspiratory oxygen concentration.

19. A device according to claim 17, wherein the control unit is configured to:
receive third information that corresponds to an oxygen concentration of a ventilation volume flow;
receive fourth information corresponding to an oxygen concentration of the first flushing volume flow;
receive fifth information corresponding to an oxygen concentration of the second flushing volume flow; and
determine the first time duration depending on the third information, on the fourth information and on the fifth information, so that by mixing the ventilation volume flow, the first flushing volume flow and the second flushing volume flow do not exceed or fall below a threshold value of the inspiratory oxygen concentration.

20. A computer program product for a device configured to flush a gas measurement line of a medical device, the computer program product comprising:
computer readable storage medium comprising a tangible device that is non-transitory; and
instructions retained and stored on the computer readable storage medium for use by an instruction execution device which cause the device and/or the medical device to execute a process for flushing the gas measurement line of the medical device, the process comprising providing a first flushing volume flow for flushing the gas measurement line in a direction of a patient during a first time interval; and interrupting the first flushing volume flow during a second time interval.
